(19) United States
(12) Patent Application Publication
(10) Pub. No.: US 2013/0323740 A1
(43) Pub. Date: Dec. 5, 2013

(54) DIRECT BLOOD ASSAY FOR DETECTION OF CIRCULATING MICRORNA IN CANCER PATIENTS

(71) Applicants: Dave S.B. Hoon, Santa Monica, CA (US); Sota Asaga, Tokyo (JP)

(72) Inventors: Dave S.B. Hoon, Santa Monica, CA (US); Sota Asaga, Tokyo (JP)

(21) Appl. No.: 13/849,397

(22) Filed: Mar. 22, 2013

Related U.S. Application Data
(63) Continuation of application No. PCT/US2011/052817, filed on Sep. 22, 2011.
(60) Provisional application No. 61/385,472, filed on Sep. 22, 2010.

(51) Int. Cl. C12Q 1/68

(52) U.S. Cl. CPC C12Q 1/6886
USPC 435/6.12; 435/6.14

(57) ABSTRACT

Methods of diagnosing, determining the progression, or determining a prognosis of a cancer in a subject are provided. Such methods may include steps of measuring a test level of one or more miR molecules in a biological sample from the subject; comparing the test level to a control level of the one or more miR molecules; and diagnosing a subject as having a cancer, differentiating between a locoregional cancer and a cancer that has progressed to a cancer with visceral or distant metastasis, or determining a prognosis for the subject having a cancer when the test level is significantly different than the control level.

### BACKGROUND

This application is a continuation of International Application No. PCT/US2011/052817, filed on Sep. 22, 2011 and now pending, which claims the benefit of U.S. Provisional Patent Application No. 61/385,472, filed Sep. 22, 2010, both of which are incorporated herein by reference in its entirety.

Breast cancer was the second leading cause of cancer death among women in the United States in 2009 (Jemal et al. 2009). Although early detection through mammographic screening has reduced breast cancer mortality (Moss et al. 2006), the sensitivity and specificity of mammography can be compromised in younger women who have dense breast tissue (Boyd et al. 2007). Minimally invasive and sensitive diagnostic approaches are needed to supplement breast imaging approaches.

There have been several attempts to develop blood biomarker assays for early breast cancer screening. Although serum based tumor biomarkers, CA15-3 and carcinoembryonic antigen (CEA) are currently used in assessment of advanced disease status, none are recommended for diagnostic use (Harris et al. 2007). Circulating tumor cells (CTC) in blood have been considered as a potential biomarker for estimating the prognostic risk of metastatic breast cancer patients (Cristofanilli et al. 2004). However, the CTC assay has limitations in the diagnosis of early breast cancer (Kahn et al. 2004), because it can only detect when breast cancer cells are being shed into circulation which is limited in early stage disease (Taback et al. 2003). At the present time, CTC can best be used as a surrogate biomarker of metastatic disease but not for early detection. In addition, the CTC assay is limited by the accuracy of retrieving CTCs from whole blood, which is a challenging requirement.

MicroRNAs (miRs) are naturally occurring small non-coding RNA molecules (18˜24 nucleotides) that interact with their target coding mRNAs to inhibit translation by promoting mRNA degradation or to block translation by binding to complementary sequences in the 3′ untranslated regions (3′ UTR) of mRNA (Du & Zamore 2005). miRs can be expressed in a tissue-specific manner and have been identified recently to play pivotal regulatory roles such as proliferation, apoptosis, and differentiation in mammalian cells (Ambros 2004; Bartel 2004; Sempere et al. 2004). miR-21 is one of the most significantly up-regulated miRs in human breast cancer, and its expression has been reported to be associated with tumor progression and poor prognosis (Si et al. 2007; Zhu et al. 2007; Frankel et al. 2008; Yan et al. 2008; Qian et al. 2009). Evidence suggests that miR-21 targets and inhibits multiple tumor suppressor genes such as TPM1 (Zhu et al. 2007), PDCD4 (Frankel et al. 2008), PTEN (Wickramasinghe et al. 2009) and other tumor-related genes.

Recently, miRs have been reported to be detected in serum or plasma and are relatively more stable than mRNA (Chim et al. 2008) in blood. Intrinsic miRs in serum were demonstrated to be stable in room temperature, can withstand multiple freeze-thaw cycles and can survive effects of RNase and DNase (Mitchell et al. 2008; Chen et al. 2008). However, the clinical utility of miR has not been investigated in a well defined cancer-related study. Therefore, it is desirable to develop a clinically useful assay for the detection of miRs and for the determination of their clinical utility.

### SUMMARY

In one embodiment, a method of detecting circulating microRNA is provided, the method comprising mixing a serum sample from a subject with a detergent; and performing a direct RT-qPCR assay without an RNA extraction step to detect a level of microRNA.

In some embodiments, methods of diagnosing a cancer in a subject are provided. Such methods may include steps of measuring a test level of one or more miR molecule in a biological sample from the subject; comparing the test level to a control level of the one or more miR molecule; and diagnosing a subject as having a cancer when the test level is significantly different than the control level.

In other embodiments, methods of determining the progression of a cancer in a subject are provided. Such methods may include steps of measuring a test level of one or more miR molecules in a biological sample from the subject; comparing the test level to a control level of the one or more miR molecules; and differentiating between a locoregional cancer and a cancer that has progressed to a cancer with visceral or distant metastasis when the test level is significantly different than the control level.

In additional embodiments, methods of determining a prognosis of a subject having a cancer are provided. Such methods may include steps of measuring a test level of one or more miR molecules in a biological sample from the subject; comparing the test level to a control level of the one or more miR molecules; and determining a prognosis for the subject having a cancer when the test level is significantly different than the control level. The prognosis may be a poor prognosis or a good prognosis, measured by a shortened survival or a prolonged survival, respectively. Further, the survival may be measured as an overall survival (OS) or disease-free survival (DFS).

In the embodiments provided above, the one or more miR molecules may include miR-16, miR-21, miR-29b or miR-210. In another embodiment, the cancer may be breast cancer or melanoma cancer. In addition, the test level and the control level are a mean C_{q} test value and a mean C_{q} control value, each of which may be normalized by an internal control.

### BRIEF DESCRIPTION OF THE FIGURES

FIG. 1 illustrates the stability of circulating miR-21. (a) The -dC_{q} (or “dC_{T}”) values of four serum samples of breast cancer patients and respective dilution samples into 2, 4, 8 fold were assessed using direct serum RT-qPCR assay. (b) Assay consistency across several freeze-thaw cycles was examined in serum samples obtained from four different breast cancer patients.

FIG. 2 illustrates the C_{q} values of circulating miR-16 in the pilot study. (a) Results of C_{q} values of circulating miR-16 by conventional RT-qPCR assay are shown. (b) Results of C_{q} values of circulating miR-16 by direct RT-qPCR assay are shown. The boxes in the figure represented between 25 and 75 percentile of distribution of values.

FIG. 3 shows a pilot study of -dC_{q} between healthy female donors and breast cancer patients; Pilot study. The comparison of -dC_{q} values representing circulating miR-21 level in healthy females and breast cancer patients with each AJCC stage. (a) Results of -dC_{q} values by conventional assay are shown. (b) Results of -dC_{q} values by direct assay are shown. The boxes in this figure represent between 25 and 75 percentile of distribution of values.

FIG. 4 shows a validation study of -dC_{q} between healthy female donors and breast cancer patients by direct serum RT-qPCR assay. Results of serum miR-21 detection by direct RT-qPCR for serum samples from 20 healthy female donors and 102 breast cancer patients are included. The boxes represent between 25 and 75 percentile of distribution of values.

FIG. 5 illustrates a differential diagnosis for breast cancer by circulating miR-21. The assessment of clinical utility of circulating miR-21 for breast cancer: (a) ROC analysis for locoregional breast cancer (AJCC stage I-III) versus healthy females by serum miR-21 expression obtained by direct RT-qPCR was presented. (b) The correlation between patients' status and test results when the cut-off value of -dC_{q} was set to 3.3. (c) ROC analysis for metastatic breast cancer (AJCC stage IV) versus locoregional breast cancer was presented. (d) The correlation between patients' status and test results when the cut-off value of -dC_{q} was set to 5.4.

FIG. 6 is a table showing the correlation between circulating miR-21 concentrations and 11 clinicopathologic characteristics of breast cancer patients.

FIG. 7 illustrates a comparison of relative miR expression levels in breast cancer T47D, MCF7 and MDA-MB-231 cell lines as indicated. The distribution chart shows each miR expression derived from miR-29a, miR-29b, miR-29c, miR-21 and miR-210.

FIG. 8 are distribution charts for miR expression levels illustrating a comparison of relative miR expression levels (miR-29a, miR-29b, miR-29c, miR-21 and miR-210) in serum samples from breast cancer patients and normal samples. The distribution charts show each miR expression derived from breast cancer patients and normal samples.

FIG. 9 shows the disease free survival (DFS) rates in patients with high miR-29b expression (bottom line) and patients with low miR-29b expression (top line). The numbers of patients with high miR-29b expression and low miR-29b expression are 51 and 50, respectively.

FIG. 10 illustrates a comparison of relative miR expression of breast cancer patients and normal samples in serum. The distribution chart shows each miR expression derived from normal samples and each TNM stage.

FIG. 11 is a table showing the correlation between circulating miR-29b concentrations and 14 clinicopathologic characteristics of breast cancer patients.

FIG. 12 is a table showing univariate and multivariate analyses of clinicopathological factors affecting disease free survival (DFS) and overall survival (OS) rate.

FIG. 13 is a distribution chart illustrating miR-210/miR-16 expression levels in plasma from metastatic melanoma patients (n=43) as compared to normal patients (n=23). The ratio of the expression of miR-210/miR-16 is significantly higher in plasma from metastatic melanoma patients (within 30 days of recurrence, n=43) compared to normal plasma (n=23). Wilcoxon p=0.0073.

FIG. 14 is a distribution chart illustrating miR-21/miR-16 expression levels in plasma from Stage III melanoma patients (n=18) versus Stage IV melanoma patients (n=20). The ratio of the expression of miR-21/miR-16 is significantly higher in plasma from stage IV melanoma patients (n=20) as compared to stage III melanoma patients (n=18). Wilcoxon p=0.0110.

### DETAILED DESCRIPTION

A direct reverse-transcription quantitative real-time polymerase chain reaction (RT-qPCR) assay for the detection of circulating microRNA molecules in a biological sample and methods for diagnosing, prognosing and analyzing a cancer are provided herein. MicroRNA (miR) molecules are a class of small non-coding RNAs whose expression changes have been associated with cancer development and progression.

In one embodiment, a direct serum assay using reverse-transcription (RT) to detect miRs without having to extract RNA, circumventing the loss of miRs in extraction steps, is provided. Efficient extraction of circulating nucleic acids from plasma or serum has been challenging in molecular detection assays, particularly when the nucleic acids are small in length, limited in the amount of nucleic acids, or limited in the amount of source material (i.e. blood).

According to some embodiments, the methods for diagnosing, prognosing and analyzing a cancer described herein may include steps of measuring a test level of one or more miR molecule in a biological sample from the subject and comparing the test level to a control level of the one or more miR molecules. The one or more miR molecules that may be measured according to the embodiments described herein may be any circulating cell-free miR molecule that is present, detected or differentially expressed in a biological sample from a subject having a cancer. In one aspect, the one or more miR molecules may be any circulating cell-free miR molecule that is present, detected or differentially expressed in a biological fluid sample (e.g., blood, plasma, serum, urine, cerebrospinal fluid) from a subject having a cancer, such as those cancers discussed below,

The results as described below demonstrate utility of the novel reverse-transcription quantitative real-time PCR (RT-qPCR) directly applied in a serum assay (“direct RT-qPCR”) to detect and quantify the concentrations of circulating miR molecules (e.g., miR-21, miR-29b, miR-210 or a combination thereof in breast cancer and melanoma cancer patients without having to extract RNA from serum. Therefore, in some aspects, the one or more miR molecules may include, but are not limited to, miR-16, miR-21, miR-29b and miR-210. In other aspects, the one or more miR molecules may be any circulating cell-free miR molecule that is present, detected or differentially expressed in a biological fluid sample (e.g., blood, plasma, serum, urine, cerebrospinal fluid) from a subject having breast cancer or melanoma cancer.

In some embodiments, the methods described herein may include a step of diagnosing a subject as having a cancer when the test level is significantly different than the control level. In other embodiments, the methods may also include a step of determining a prognosis for a subject having a cancer when the test level is significantly different than the control level. The prognosis may be a poor prognosis or a good prognosis, as measured by a decreased length of survival or a prolonged (or increased) length of survival, respectively. Further, the survival may be measured as an overall survival (OS) or disease-free survival (DFS). In some aspects, a diagnosis or a prognosis of cancer may be made when the test level is significantly higher than the control level or significantly lower than the control level. According to some embodiments, a diagnosis of cancer or a poor prognosis may be made when the test levels of miR-21, miR-29b, miR-210 or a combination thereof are significantly higher than a control level (or “an increased test level”). However, in other embodiments, other miR molecules and corresponding test levels may be identified that are significantly lower than control levels (or “a decreased test level”) in samples from subjects having cancer.

The methods described herein may also be used to differentiate between a locoregional cancer (i.e., an AJCC stage I-III cancer) and a cancer that has progressed to a cancer with visceral or distant metastasis (i.e., an AJCC stage IV cancer) when the test level is significantly different than the control level.

A “test” level, expression level or other calculated test level of an miR molecule or other biomarker refers to an amount of a biomarker, such as an miR molecule, in a subject's undiagnosed biological sample. The test level may be compared to that of a control sample, or may be analyzed based on a reference standard that has been previously established to determine a status of the sample. Such a status may be a diagnosis, prognosis or evaluation of a disease or condition. In one embodiment, the disease is a cancer, disease or condition. A test sample or test amount can be either in absolute amount (e.g., nanogram/mL or microgram/mL) or a relative amount (e.g., relative intensity of signals).

A “control” level, expression level or other calculated level of an miR molecule or other biomarker of a marker can be any amount or a range of amounts to be compared against a test amount of a marker. For example, a control amount of a marker can be the amount of a marker in a population of patients with a specified condition or disease (e.g., malignancy, cancer or non-cancerous lung disease or condition) or a control population of individuals without said condition or disease. A control amount can be either in absolute amount (e.g., nanogram/mL or microgram/mL) or a relative amount (e.g., relative intensity of signals).

In some embodiments, the test level and the control level may be expressed as a mean C_{q} test value and a mean C_{q} control value as described further below. The mean C_{q} test value and a mean C_{q} control value are normalized by an internal control (e.g., miR-16 and RNU6B).

An “increase or a decrease” or a difference in the test level of a gene product compared to a preselected control level as used herein refers to an over-expression or an under-expression as compared to the control level. In some embodiments, an increase or decrease is typically significantly different if said increase or decrease has a p value of less than 0.5, or less than 0.05 (p<0.5 or p<0.05).

An miR molecule or other biomarker that is either over-expressed or under-expressed can also be referred to as being “differentially expressed” or as having a “differential level.” According to the methods described herein, a diagnosis of cancer may be made based on the detection of one or more miR molecules associated with the one or more miR molecules that are differentially present or differentially expressed in a biological sample. The phrase “differentially present” or “differentially expressed” refers to a difference in the quantity or intensity of a marker present in a sample taken from patients having a cancer as compared to a comparable sample taken from patients who do not have the cancer. For example, an miR molecule is differentially expressed between the samples if the amount of the miR molecule in one sample is significantly different (i.e., p<0.05) from the amount of the miR molecule in the other sample. It should be noted that if the miR molecule or other marker is detectable in one sample and not detectable in the other, then the miR molecule can be considered to be differentially present.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased biological fluids, normal and diseased cell-free biological fluids, normal and diseased cells, or among cells which have undergone different disease events or disease stages. Further, a gene that is differentially expressed in one type of biological sample may or may not be indicative of its presence or expression in another type biological sample. For example, a gene that is differentially expressed in a tumor tissue is not necessarily indicative of its presence in a blood or other biological fluid sample.

Any of the methods and examples described herein may be referred to as either “diagnosing” or “evaluating” cancer: initially detecting the presence or absence of cancer; determining a specific stage, type or sub-type, or other classification or characteristic of cancer; determining whether a tumor is a benign lesion or a malignant tumor; or determining/monitoring cancer progression (e.g., monitoring tumor growth or metastatic spread), remission, or recurrence.

“Diagnose,” “diagnosing,” “diagnosis,” and variations thereof refer to the detection, determination, or recognition of a health status or condition of an individual on the basis of one or more signs, symptoms, data, or other information pertaining to that individual. The health status of an individual can be diagnosed as healthy or normal (i.e., a diagnosis of the absence of a disease or condition) or diagnosed as ill or abnormal (i.e., a diagnosis of the presence, or an assessment of the characteristics, of a disease or condition). The terms “diagnose,” “diagnosing,” “diagnosis,” or other analogous terms encompass, with respect to a particular disease or condition, the initial detection of the disease; the characterization or classification of the disease; the detection of the progression (e.g., the stage of a cancer), remission, or recurrence of the disease; and the detection of disease response after the administration of a treatment or therapy to the individual.

“Prognose,” “prognosing,” “prognosis,” and variations thereof refer to the course of a disease or condition in an individual who has the disease or condition (e.g., patient survival), and such terms encompass the evaluation of disease response after the administration of a treatment or therapy to the individual. A biomarker, such as an miR molecule that is differentially expressed or detected in a biological sample as described herein, may be a prognostic or a predictive biomarker. Prognostic and predictive biomarkers are distinguishable. A prognostic biomarker may be associated with a particular condition or disease, but is based on data that does not include a non-treatment or non-diseased control group. A predictive biomarker is associated with a particular condition or disease, as compared to a non-treated, non-diseased or other relevant control group (e.g., a different stage or cancer). By including such a control group, a prediction can be made about the prognosis of a patient that can not be made using a prognostic biomarker.

“Evaluate,” “evaluating,” “evaluation,” and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses monitoring or assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” cancer can include, for example, any of the following: prognosing the future course of cancer in an individual; predicting the recurrence of cancer in an individual who apparently has been cured of cancer (e.g., by surgical resection); or determining or predicting an individual's response to a cancer treatment or selecting a cancer treatment to administer to an individual based upon a determination of the miR levels, values or expression levels derived from the individual's biological sample.

The methods described herein may be used to diagnose, prognose or analyze any type of tumor type or cancer. The terms “malignancy,” “cancer” and “cancerous” refer to or describe the physiological condition in mammals that is typically characterized by unregulated cell growth. Cancers and tumor types that may be treated or attenuated using the methods described herein include but are not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, prostate cancer, rectal cancer, renal cancer, sarcoma, testicular cancer, thyroid cancer, and uterine cancer. In addition, the methods may be used to treat tumors that are malignant (e.g., primary or metastati cancers) or benign (e.g., hyperplasia, cyst, pseudocyst, hematoma, and benign neoplasm).

“Biological sample,” “sample,” and “test sample” are used interchangeably herein to refer to any material, biological fluid, tissue, or cell obtained or otherwise derived from an individual including, but not limited to, blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, milk, bronchial aspirate, synovial fluid, joint aspirate, cells, a cellular extract, and cerebrospinal fluid. This also includes experimentally separated fractions thereof. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” may also include materials containing homogenized solid material, such as from a stool sample, a tissue sample, or a tissue biopsy. The term “biological sample” also includes materials derived from a tissue culture or a cell culture. Further, it should be realized that a biological sample can be derived by taking biological samples from a number of individuals and pooling them or pooling an aliquot of each individual's biological sample. The pooled sample can be treated as a sample from a single individual and if the presence of cancer is established in the pooled sample, then each individual biological sample can be re-tested to determine which individuals have cancer.

The miR molecules may be measured and/or quantified by any suitable method known in the art including, but not limited to, reverse transcriptase-polymerase chain reaction (RT-PCR) methods, microarray, serial analysis of gene expression (SAGE), gene expression analysis by massively parallel signature sequencing (MPSS), immunoassays such as ELISA, immunohistochemistry (IHC), mass spectrometry (MS) methods, transcriptomics and proteomics. In one embodiment, the method of measuring an expression level includes performing RT-qPCR without an RNA extraction step. In one embodiment, the method of detecting and measuring one or more circulating miRNA molecules is provided, the method comprising performing a direct RT-qPCR assay on a biological sample without an RNA extraction step to detect a level of microRNA. The direct RT-qPCR assay may include a step of mixing the serum sample a detergent (e.g., Tween20). The microRNA may be any relevant microRNA including, but not limited to miRNA-16, miRNA-21, miRNA-29b, miRNA-210 or a combination thereof.

Efficiency of miR isolation from blood and analysis by PCR has been a significant limitation in developing efficient miR blood assays. Therefore, the methods described herein using a direct serum RT-qPCR assay are clinically useful and relevant for the detection of circulating miR molecules. Circulating miRs in blood have been found in free form (Mitchell et al. 2008) or encapsulated in exosomes (Ng et al. 2009; Zhu et al. 2009; Taylor et al. 2008). There has been little information about the structure of exosome involving miR, and not all exosomes contain miR molecules. Therefore, the efficacy of assaying exosomes to measure miR has been limited. Cancer derived exosomes are soluble in detergents (Hunter et al. 2008). Therefore, according to the embodiments described herein, a suitable detergent (e.g., Tween 20) may be used in the direct serum assay for measuring and assessing potential serum miRs, regardless of whether they were lipid bound or from exosomes, to improve PCR efficacy. Tween 20 and other suitable detergents can dissociate lipid bound nucleic acids in serum. As described further in the Examples below, the direct serum RT-qPCR assay was demonstrated to be effective and robust for detecting circulating miR. Moreover, the direct serum RT-qPCR assay has at least the following advantages over conventional RT-qPCR assay: (1) elimination of miR loss during the extraction step, (2) streamlines assay procedures, (3) minimizes both human and mechanical errors, and (4) reduces time and overall cost.

Assays for cell-free (or “extracellular”) circulating nucleic acids should use an internal reference control in the fluid being sampled. An internal control for circulating miR should be a nucleic acid in the serum that can be consistently detected, the level of which is not influenced by patient's disease status. In some embodiments, miR-16 may be used as an internal control for circulating miRs. Results using conventional RT-qPCR and direct serum RT-qPCR confirmed that miR-16 was consistently detected in serum and may be used as an internal control reference marker (or “control reference marker”) for the direct serum assay. Without a control reference marker, negative results are not distinguishable from false negatives. Thus, use of a control reference marker is important in the assessment of cell-free nucleic acids in blood, serum, plasma or any other biological fluid.

As discussed above, the direct RT-qPCR assay was developed for detection of circulating nucleic acids (e.g., miR molecules). In one embodiment, serum was assessed by direct RT-qPCR for detection of circulating miR-21 in patients of different stages of breast cancer and healthy female donors to determine sensitivity and specificity. The direct serum RT-qPCR assay significantly discriminated circulating miR-21 levels in different stage breast cancer patients (n=102) from healthy females (n=20). Patients with distant metastatic breast cancer were distinguished from locoregional breast cancer with high sensitivity and specificity. For discrimination of locoregional breast cancer patients from healthy donors, odds ratio was 1.796 and the AUC was 0.721. Breast cancer patients with high circulating miR-21 correlated significantly (p<0.001) to visceral metastasis in a multivariate analysis compared with other clinicopathological prognostic factors. The direct serum-RT-qPCR assay provides a novel approach in the accurate assessment of circulating miR without extraction of RNA from serum in patients. The detection of circulating miR-21 in serum demonstrates clinical utility for diagnosis and detection of breast cancer progression.

The detection of circulating miR-21 in serum obtained from breast cancer patients by the direct serum RT-qPCR assay described herein was investigated for potential clinical utility. This direct serum assay demonstrated that circulating miR-21 was significantly up-regulated in locoregional breast cancer patients compared to healthy female donors and in metastatic breast cancer patients compared to locoregional breast cancer patients. This demonstrates the utility of a direct serum RT-qPCR assay for assessing circulating miR. In addition to the technique used to directly detect circulating miR in serum by RT-qPCR, it was also demonstrated that circulating miR-21 levels may be used to detect early stage and progression of breast cancer.

In other embodiments, the direct RT-qPCR may be used to detect other circulating miR molecules that are differentially expressed and detected in biological samples. For example, elevated expression levels (or test levels) of miR-21, miR29b, miR210 or a combination thereof in breast tumors and melanoma tumors are associated with breast cancer and melanoma cancer diagnosis and progression, as described in detail in the Examples below, In addition, although the direct RT-qPCR assay was initially developed for measuring circulating, cell-free miR molecules, the assay may also be used to measure extracellular or cell-free miR molecules or other nucleic acid molecules in any other biological fluid including, but not limited to, whole blood, plasma, urine, lymph fluid, cerebrospinal fluid, or any other suitable biological sample referred to herein.

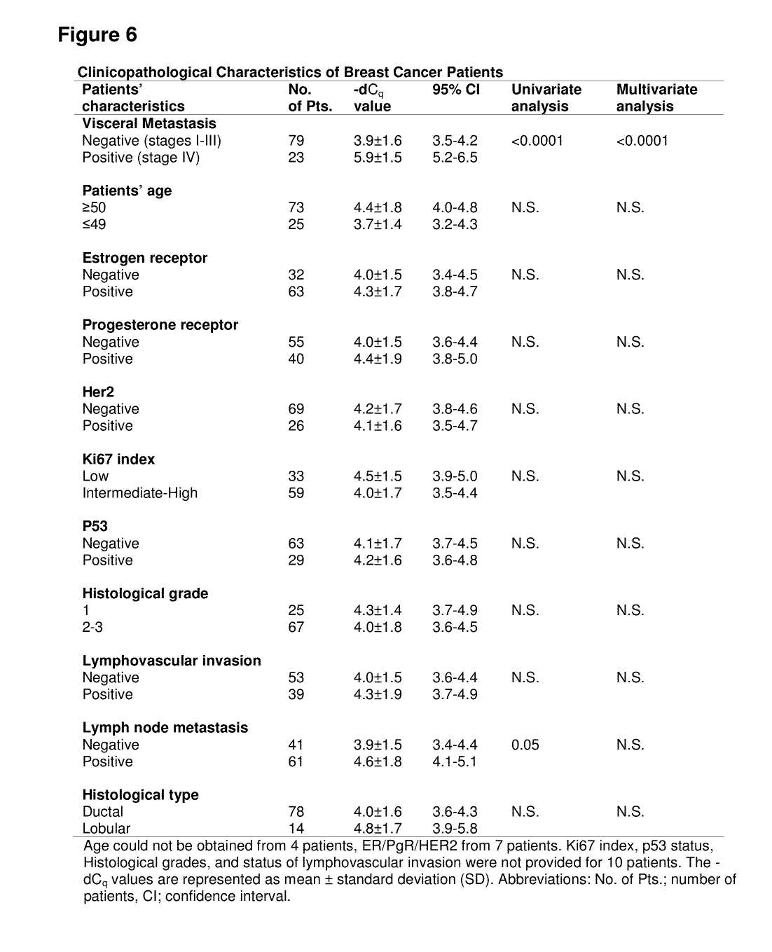

Several miR molecules may be associated with cancer. For example, miR-21 has been found to stimulate cell invasion and metastasis in different tumors (Ambros 2004) including breast cancer as demonstrated by in vitro and in vivo assays, and this ability was partially explained by its direct repression of maspin, PDCD4, and urokinase plasminogen activator surface receptor (Gibbings et al. 2009). Moreover, there have been several reports that miR-21 expression in breast tumor was correlated with advanced clinical stage, lymph node metastasis, and poor prognosis in breast cancer (Yan et al. 2008; Qian et al. 2009). A recent report that studied the utilization of circulating miRs as cancer biomarkers showed that circulating miR-195 increased in pre-operative breast cancer patients while it decreased in post-operative breast cancer patients and that specific circulating miRs were correlated with certain clinicopathological variables (Gastpar et al. 2005). The conventional assay was performed as part of the pilot study to demonstrate the ability to detect miR and to compare it to the direct serum RT-qPCR assay. As described below, the conventional RT-qPCR assay was unable to discriminate patients with locoregional breast cancer from those with metastatic breast cancer, whereas the direct serum assay was capable of doing so. The direct serum assay successfully demonstrated that the level of circulating miR-21 is related to AJCC stage of breast cancer, although the relationship between circulating miR-21 and patients' estrogen receptor (ER) status should be explored further (See FIG. 6 below).

Mammography is the primary choice for breast cancer screening today. Recently, the U.S. Preventive Services Task Force recommended against routine mammography screening in women aged 40 to 49 (U.S. Preventative Services Task Force, Ann Intern Med 2009; 151:738-47). Biennial mammography screening expanding to women ages 40 to 69 years reduced mortality only by 3% compared to ages 50 to 69, yet consumes considerable resources and yields false-positive results (Mandelblatt et al. 2009). The multivariate analysis described below showed that patient's age did not affect the circulating miR-21 level which further validates the clinical value of circulating miR-21 for breast cancer detection regardless of age.

The findings described below show that the level of circulating miR-21 is correlated with AJCC staging and is independent of ER or age. Therefore, circulating miR-21 may be a potential biomarker for breast cancer progression and detection to improve diagnosis.

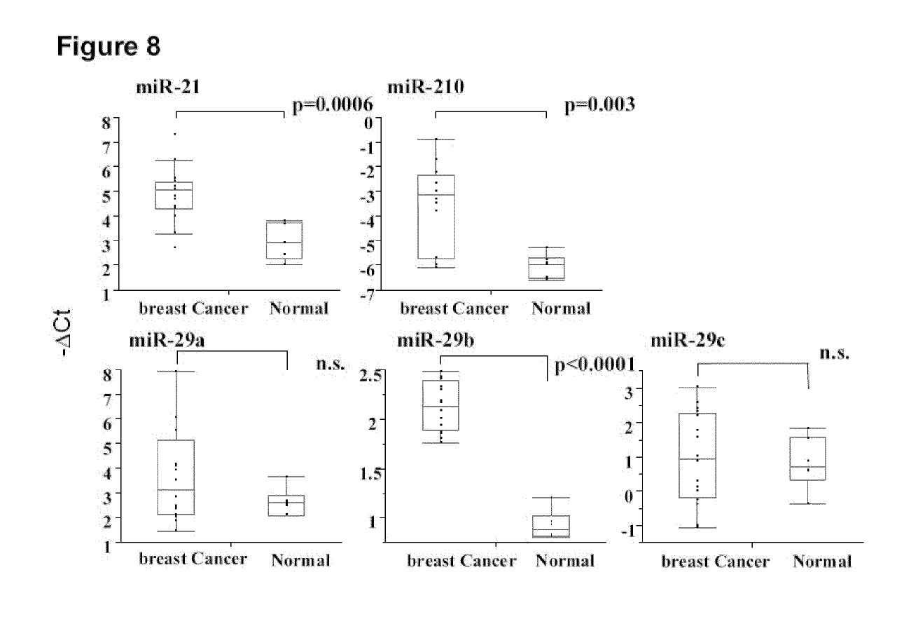

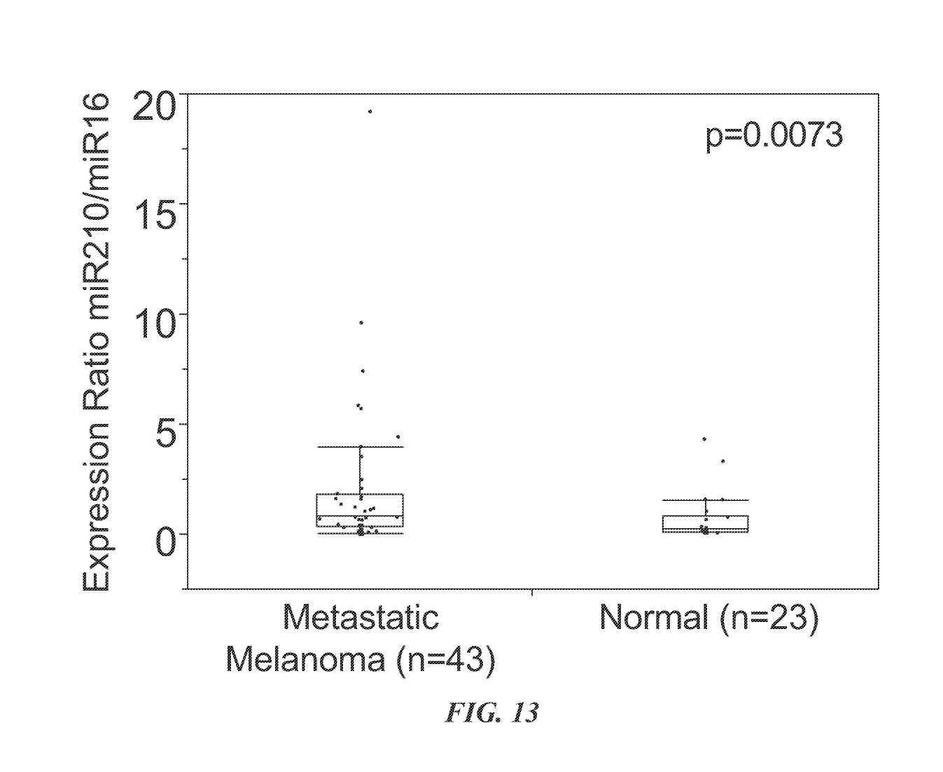

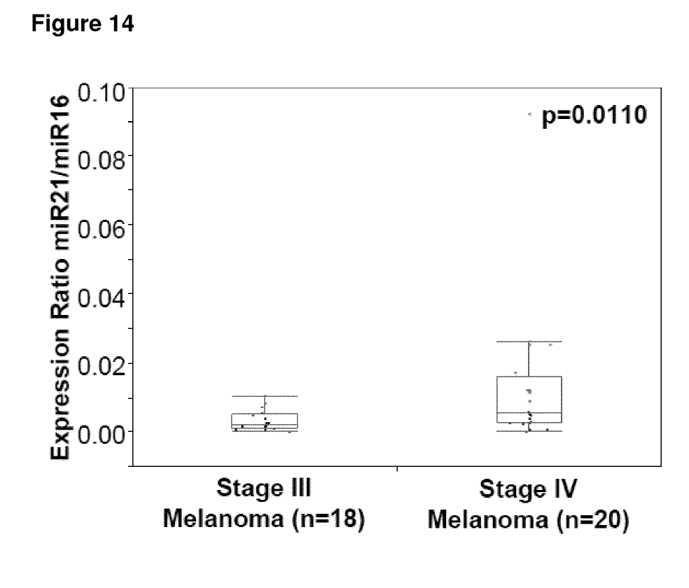

The level of circulating miR-21, miR29b and miR210 are elevated in serum of breast cancer patients and may be used as a diagnostic serum biomarker in a clinically defined population of breast cancer patients. As discussed in the Examples below, levels of circulating miR-21, miR29b and miR210 in serum are significantly higher in breast cancer patients compared to healthy female controls (FIG. 8). Further, levels of circulating miR-21, miR29b and miR210 (normalized to miR-16) are significantly higher in (i) metastatic melanoma cancer patients as compared to healthy female controls (FIG. 13); and (ii) Stage IV melanoma as compared to Stage III melanoma (FIG. 14). Circulating miR-21 levels distinguish patients with locoregional breast cancer from healthy females and further distinguish patients with distant metastases from locoregional disease. The level of circulating miR-21 may be an important blood biomarker for breast cancer screening and may be used as a biomarker for progression and diagnosis of distant metastasis.

A direct PCR assay has been established to study circulating DNA in blood from patients with breast cancer and other cancers (Umetani et al. 2006a; Umetani et al. 2006b). This type of direct assay demonstrates that the integrity of circulating DNA as measured by a direct serum PCR assay for ALU repeats was useful in detecting progression of breast and gastrointestinal cancers. The Examples below show that another direct serum assay approach may be used to detect miRs in the blood.

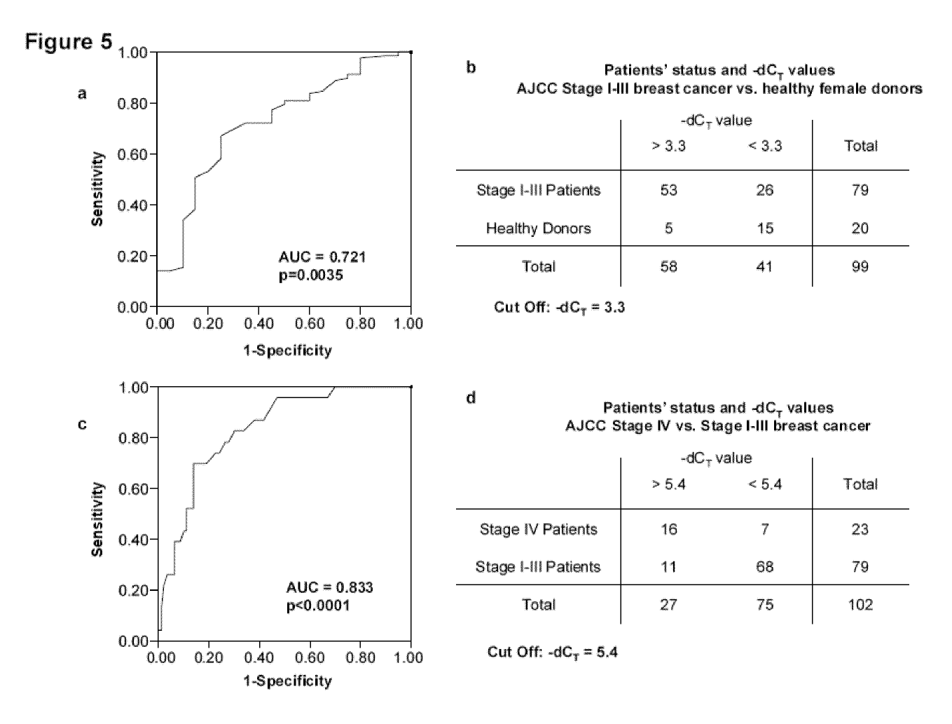

To determine their diagnostic performance, a receiver operating characteristic (ROC) curve was generated for each significant miR molecule identified herein. A “receiver operating characteristic (ROC) curve” is a generalization of the set of potential combinations of sensitivity and specificity possible for predictors. A ROC curve is a plot of the true positive rate (sensitivity) against the false positive rate (1-specificity) for the different possible cut-points of a diagnostic test. FIGS. 5A and 5C are graphical representations of the functional relationship between the distribution of a biomarker's or a panel of biomarkers' sensitivity and specificity values in a cohort of diseased subjects and in a cohort of non-diseased subjects. The area under the curve (AUC) is an overall indication of the diagnostic accuracy of (1) a biomarker or a panel of biomarkers and (2) a receiver operating characteristic (ROC) curve.

Having described the invention with reference to the embodiments and illustrative examples, those in the art may appreciate modifications to the invention as described and illustrated that do not depart from the spirit and scope of the invention as disclosed in the specification. The Examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### EXAMPLE 1

### Clinical Relevance of Serum miR-21, miR-29b and miR-210 in Breast Cancer Patients

### Patients, Cells and Methods

Paraffin-embedded archival tissue (PEAT) analysis. Paraffin-embedded archival tissue (PEAT) samples of primary tumor and adjacent normal breast were obtained from 14 patients who underwent surgical treatment for invasive breast cancer at JWCI at Saint John's Health Center (SJHC) in 2000-2007. Patients had American Joint Committee on Cancer (AJCC) stage I (N=4), stage II (N=1), stage III (N=5), or stage IV (N=4) disease. All tissue specimens for this study were obtained according to protocol guidelines set forth by JWCI and approved by the Western Institutional Review Board.

Serum samples for pilot and validation study. Blood samples collected in red tiger top gel separator tubes (Fisher Scientific) from patients or healthy donors were processed within 2-5 hours as follows: the serum was separated by centrifugation and passed through a 13-mm serum filter (Fisher Scientific) to remove potential contaminating cells as previously described (Umetani et al. 2006a). Serum was divided into aliquots and immediately cryopreserved at −80° C. For the pilot study, serum samples were obtained from 10 healthy female donors and 40 women with pathologic (AJCC) stage I (N=10), II (N=10), III (N=10) or IV (N=10) breast cancer. The 40 patients included all 14 patients in the PEAT study. For the validation study, serum samples were obtained from an additional 10 healthy women and 62 women with AJCC stage I (N=21), stage II (N=16), stage III (N=12), or stage IV (N=13) breast cancer. All patients with AJCC stage III disease had lymph node metastasis; and all patients with AJCC stage IV disease had visceral metastasis. All patients underwent surgical treatment for invasive breast cancer in 2000-2007 at SJHC. All serum specimens for this study were obtained according to institutional review board (IRB) approved protocol and after the sample donors provided informed consent.

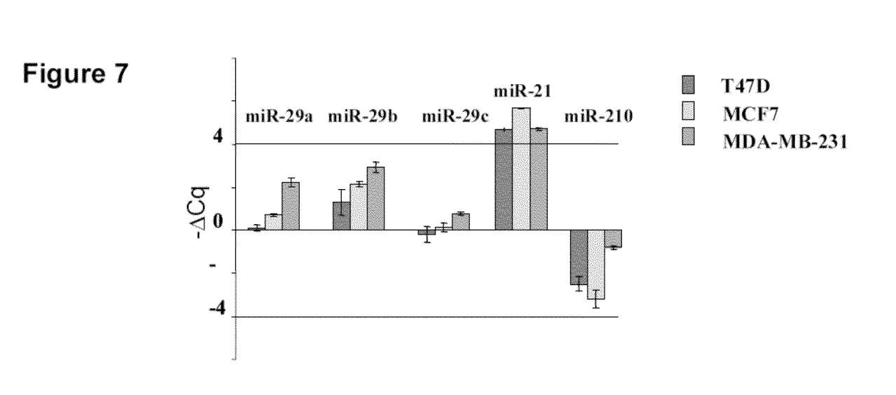

Cell culture. T47D, MCF7 and MDA-MB-231 breast cancer cell lines were cultured according to standard conditions. The cell lines were used to establish relative miR expression levels (FIG. 7).

RNA extraction from PEAT specimens. Total RNA was extracted from 500 μL of serum by using TRI reagent BD (Molecular Research Center). Ten sections, each 10 μm thick, were cut from each PEAT block. Deparaffinized tissue sections were digested using proteinase K, and RNA was extracted using a modified protocol of the RNAWiz Isolation Kit (Applied Biosystems, Foster City, Calif.) (Takeuchi et al. 2004). The RNA was quantified and assessed for purity using UV spectrophotometry and the Quant-iT RiboGreen RNA Assay kit (Invitrogen, Carlsbad, Calif.) (Takeuchi et al. 2004).

Conventional qRT-PCR assay. Total RNA was extracted from 500 μ1 of serum from breast cancer patients and healthy female donors using TRI reagent BD (Molecular Research Center INC., Cincinnati, Ohio) for conventional qRT-PCR. Ten ng of total RNA extracted from tissue and serum samples was dissolved in 5 uL H₂O (2 ng/uL) for reverse transcription using miR-specific RT primers (Exiqon, Denmark). The transcribed specific cDNA was first diluted tenfold by molecular grade H₂O to a total of 100 uL of cDNA from 10 ng of total RNA, then 2.5 uL of cDNA was used as the PCR template in each reaction. miR-specific, Locked Nucleic Acid (LNA)-based forward primer and universal reverse primer (Exiqon) were used for each PCR reaction. Forty-five PCR cycles at 60° C. annealing temperature were performed, and all samples were assessed in duplicates. RNU6B was used as an internal control for the tissue studies, and miR-16 was used for the serum studies.

PerfeCTa™ SYBR Green Super Mix for iQTM (Quanta Bioscience, Gaithersburg, MD) and iCycler real-time PCR instrument (Bio-Rad, Hercules, Calif.) were utilized for all real-time PCR with melting curve analysis. Target amplification was normalized with the internal control, and comparative quantification is recorded as the -dC_{q} (or “dC_{T}”). In PEAT, the difference of threshold cycle (C_{q}) values obtained for the target miR and internal control in a cancer specimen was compared to the difference of the C_{q} values obtained in adjacent normal breast tissue. For the serum studies, comparison of the difference of C_{q} values between target miR and internal control was performed. The results from clinicopathological subgroups of patients were compared.

Direct serum RT-qPCR assay. In the direct serum assay, only a small aliquot of the serum was needed for the RT-qPCR reaction. To deactivate or solubilize proteins that might inhibit RT-qPCR reaction, 2.5 μL of each serum sample was mixed with 2.5 μL of a preparation buffer that contained 2.5% Tween 20 (EMD Chemicals, Gibbstown, N.J.), 50 mmol/L Tris (Sigma-Aldrich, St. Louis, Mo.), and 1 mmol/L EDTA (Sigma-Aldrich). 5 μL RT reagent mixture containing the same RT reagents used for RT-qPCR with extracted RNA is added directly to 5 μL of the serum in preparation buffer and incubated in 37° C. for 2 hrs, followed with a 5 minute enzyme inactivation step at 95° C. The transcribed cDNA was diluted tenfold by H₂O and then centrifuged at 9000 g for 5 min to eliminate the protein precipitant. 2.5 μL of the supernatant cDNA solution was used as template for qPCR. The qPCR conditions, primers, reagents and data analysis used were the same as those described in the RT-qPCR with extracted RNA section above.

Biostatistical Analysis. The correlation of -dC_{q} values between conventional and direct serum RT-qPCR were measured by Pearson correlation coefficient. The differences of -dC_{q} values which represent levels of circulating miR-21 detected were compared among different groups using Student-Newman-Keuls Test, and P values <0.05 are considered significant. Ryan-Einot-Gabriel-Welsch Multiple Range Test and Tukey's Honestly Significant Difference Test were used along with Student-Newman-Keuls Test in pairwise comparison of conventional and direct serum assay in different groups. In differentiating locoregional breast cancer from healthy females and metastatic breast cancer from locoregional breast cancer by circulating miR-21, Logistic Regression analysis was used and receiver operating characteristics (ROC) curves and their area under curve (AUC) values are reported. The General Linear Model (GLM) procedure was used as a multivariate analysis in identifying clinicopathological factors significantly associated with miR-21 level.

### Results

Breast tissue analysis of miR-21. Analysis of PEAT for miR-21 confirmed its up-regulation in breast cancer tissues using the optimized RT-qPCR assay. Ten ng total RNA from each PEAT sample was analyzed using RT-qPCR. The mean C_{q} value (95% Confidence Interval (CI)) of the target miR (miR-21), was 19.2 (18.7-19.8) in breast cancer tissue PEAT and 22.5 (21.6-23.4) in normal breast tissue PEAT. The mean C_{q} value (95% CI) of internal control, RNU6B, was 23.8 (22.9-24.6) in breast cancer tissues and 25.1 (24.2-26.0) in normal breast tissues. The comparative miR-21 expression in tumor tissue as measured by the difference of dC_{q} (ddC_{q}) from the tumor and the adjacent normal tissues and the ddC_{q}s were between 0.2 and 3.9 (95% CI 1.3-2.6). [ddC_{q}=(C_{q miR21 normal}−C_{q RNU6B normal})−(C_{q miR21 cancer}−C_{q RNU6B cancer})] This demonstrated that the RT-qPCR assay described herein can robustly detect up-regulation of miR-21 levels in PEAT breast cancer as compared to normal breast tissue.

Optimization of direct serum RT-qPCR assay. A direct serum assay for detecting circulating DNA was previously established , but it was previously not determined whether a direct assay could be used to detect circulating RNA or miRNA molecules using a reverse transcriptase PCR method. First, it was determined whether a surfactant, Tween 20, together with proteinase K , can be applied in the direct RT-qPCR assay. Next, the following combinations of Tween 20 (T) and 1 ug/uL proteinase K (K) in the preparation buffer were tested: (A) no T or K, (B) K only, (C) 1.0% T and K, (D) 2.5% T and K, (E) 1.0% T only, and (F) 2.5% T only treatment. Serum samples from a training set of 12 breast cancer patients, later included in the pilot study, were used; and results were compared to those for RT-qPCR with RNA extracted from serum. No miRs were detected using combinations A through D. Combination E showed improved sensitivity, but no linear correlation (r=−0.064) to RT-qPCR with RNA extracted from serum. In contrast, combination F showed a linear correlation (r=0.796) to RT-qPCR with RNA. Thus, serum with the addition of 2.5% Tween 20 was selected for subsequent pilot and validation studies using direct serum and analyzed using RT-qPCR. These studies demonstrated that circulating miR may be assessed directly from serum, bypassing the tedious extraction of miR which is prone to generate inaccurate assessment and false negative results.

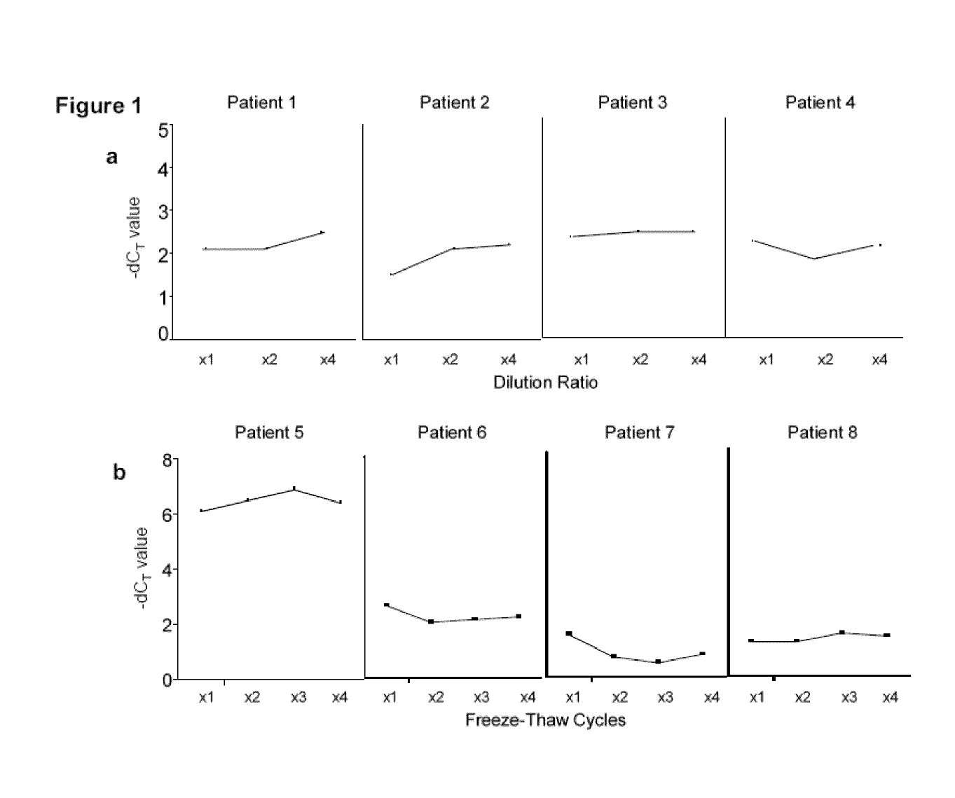

Direct serum RT-qPCR assay robustness. A serum dilution study was carried out in order to demonstrate that the variation of total RNA in serum did not affect the results of -dC_{q} values by direct serum RT-qPCR assay. The results of -dC_{q} obtained from diluting sera 2 and 4 fold were compared to the results from undiluted samples. Serum samples from four representative AJCC stage III breast cancer patients were used for this study. There was no significant difference in -dC_{q} values for miR-21 standardized by miR-16 across the two dilution groups and undiluted group (FIG. 1a).

Stability of miR in serum was investigated by performing direct serum RT-qPCR assay on four randomly selected serum samples selected from the study patient group including AJCC stage III breast cancer patients, which were subjected to four freeze-thaw cycles between −80° C. and 23° C. There was no significant difference in -dC_{q} values for miR-21 across the four freeze-thaw cycles (FIG. 1b).

Comparison of the direct serum and conventional RT-qPCR assays. After establishing a robust, reproducible and optimal direct serum RT-qPCR assay without RNA extraction, a pilot study was performed to compare the direct serum RT-qPCR assay to the conventional RT-qPCR assay requiring RNA extraction. A total of 50 serum samples from 10 healthy donors and 40 breast cancer patients with AJCC stage I-IV (10 patients of each stage) were utilized in the study.

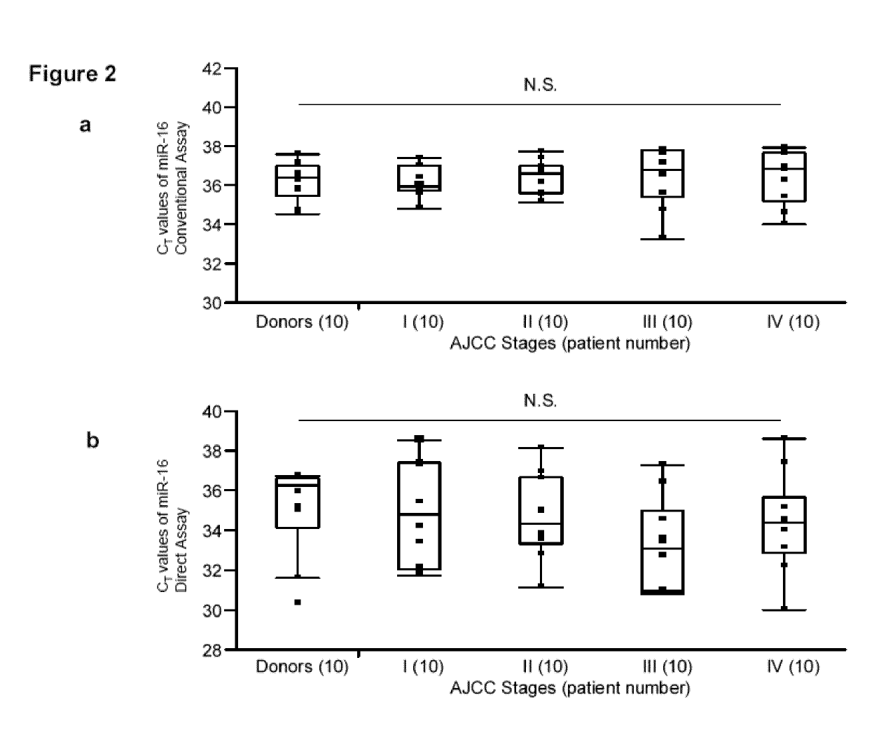

The mean C_{q} values (95% CI) of miR-16 by conventional assay were 36.2 (35.5-36.9) in healthy donors, and 36.2 (35.5-36.9), 36.4 (35.7-37.2), 36.4 (35.7-37.1), and 36.4 (35.7-37.1) in AJCC stage I, II, III, and IV breast cancer patients, respectively (FIG. 2a). The direct serum assay demonstrated that mean C_{q} values (95% CI) of miR-16 were 35.1 (33.5-36.8) in healthy donors, and 34.9 (33.1-36.8), 34.6 (33.1-36.1), 33.2 (31.5-34.8), and 34.4 (32.6-36.1) in AJCC stage I, II, Ill, and IV breast cancer patients, respectively (FIG. 2b). Both assays demonstrated no significant difference in miR-16 C_{q} values among healthy donors and all breast cancer stage groups. These results support that miR-16 is present in serum at a consistent level, and it could be used as an internal control to normalize sampling and PCR variations in both conventional and direct serum RT-qPCR assay.

The conventional assay demonstrated that the mean -dC_{q} values (95% CI), that is the difference of C_{q} values between miR-16 and miR-21, representing circulating miR-21 detection levels were 3.9 (3.1-4.7) in healthy donors, and 6.3 (5.6-7.0), 6.0 (5.3-6.8), 5.9 (5.1-6.7), and 7.0 (5.8-8.2) in AJCC stage I, II, III, and IV respectively. The mean -dC_{q} values (95% CI) by the direct serum assay were 1.8 (0.8-2.7) in healthy donors, and 4.0 (3.3-4.6), 3.6 (3.0-4.2), 3.4 (3.0-3.9), and 5.0 (4.2-5.7) in AJCC stage I, II, Ill, and IV respectively. There was a significant linear correlation in -dC_{q} values between both assays (r=0.796).

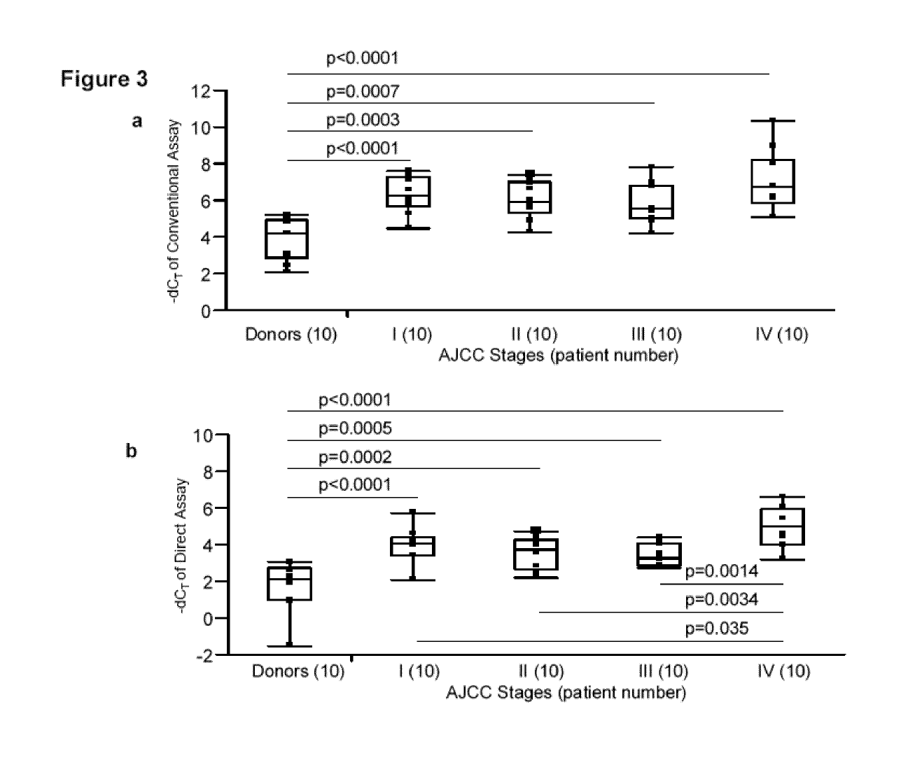

The conventional RT-qPCR assay demonstrated that the differences in -dC_{q} were significant between healthy female donors and breast cancer patients, whereas no significant difference was observed among breast cancer stages (FIG. 3a). The direct serum RT-qPCR assay showed that the differences in -dC_{q} were significant not only between healthy female donors and breast cancer patients but also significant between patients with locoregional breast cancer (AJCC stage I-III) and metastatic breast cancer (AJCC stage IV) (FIG. 3b). The same results were obtained using three different statistical procedures, Student-Newman-Keuls Test, Ryan-Einot-Gabriel-Welsch Multiple Range Test, and Tukey's Honestly Significant Difference Test.

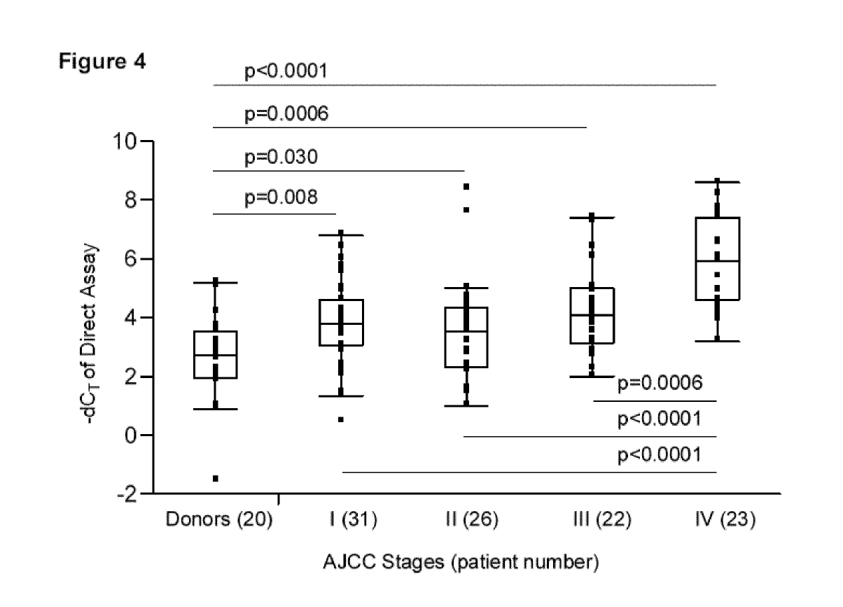

Clinical utility of circulating miR-21. Based on the results of the 50 subject pilot study, the direct RT-qPCR assay was used to validate the clinical utility of circulating miR-21 level for breast cancer. In serum analysis of all patients studied (pilot and validation groups) consisting of 20 healthy females and 102 breast cancer patients, the mean -dC_{q} values (95% CI) were 2.6 (1.9-3.3) in healthy donors and 3.8 (3.3-4.3), 3.6 (2.9-4.3), 4.3 (3.6-5.0), and 5.9 (5.2-6.5) in patients with stages I (n=31), II (n=26), III (n=22), and IV (n=23) breast cancer, respectively. The miR-21 detection level was significantly lower in healthy donors compared to breast cancer patients with any stage of disease (FIG. 4). Furthermore, circulating miR-21 levels were significantly higher in metastatic breast cancer patients than locoregional breast cancer patients (FIG. 4).

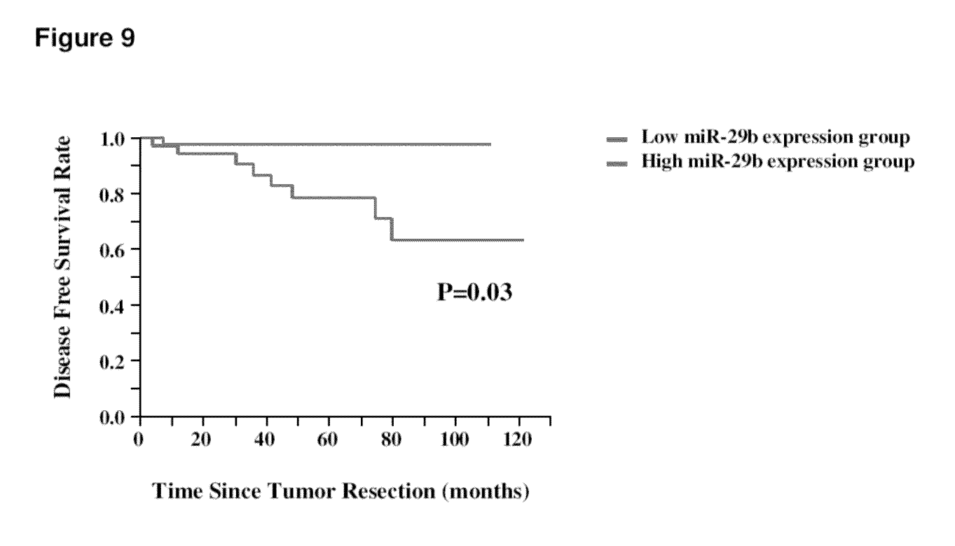

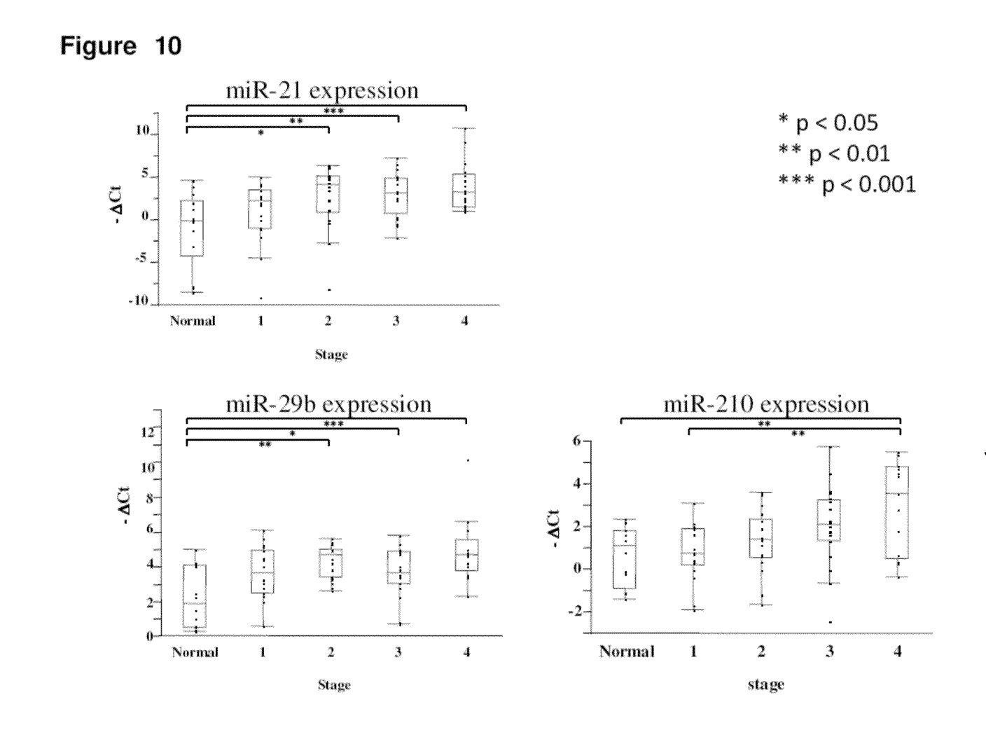

Clinical utility of circulating miR-29b and miR-210. The direct RT-qPCR assay was also used to validate the clinical utility of circulating miR-21, miR-29b and miR-201 levels for breast cancer (FIGS. 9-10). -dCt levels for miR-21, miR-29b and miR-210 were all significantly higher in breast cancer patients than normal patients, whereas miR-29a and miR-29c were not significant even though they were detected in patients, (FIG. 8). This indicates that individual members of an miRNA family do not necessarily share the same role as a biomarker for a disease or condition. Further, a significant trend of increasing -dCt levels in each of the miR molecules is shown as the cancer progresses (FIG. 10). A oneway analysis of ddCt for miR210 (target) and miR16 (reference) in breast serum showed significant differences between the following different stages of cancer: (i) Normals were significantly different from Stage III (p-Value <0.0001); (ii) Normals were significantly different from Stage IV (p-Value 0.0003); (iii) Stage I was significantly different from Stage III (p-Value 0.0022); (iv) Stage I was significantly different from Stage IV (p-Value 0.0138); and (v) Stage II was significantly different from Stage III (p-Value 0.0132).

ROC analysis was performed to assess sensitivity and specificity of the direct serum RT-qPCR assay. For discrimination of locoregional breast cancer patients from healthy donors, odds ratio was 1.796 (95% CI 1.213-2.661) and the AUC was 0.721 (FIG. 5a). When the cut-off value was set to the optimal point, 3.3, specificity was 75%, sensitivity was 67%, and positive predictive value was 91% (FIG. 5b). It was also determined whether circulating miR-21 could discriminate between patients with visceral metastasis from patients with locoregional breast cancer. The ROC results demonstrated that odds ratio was 2.153 (95% CI 1.514-3.062) and AUC was 0.833 (FIG. 5c). When the cut-off value was set to optimal point, 5.4, specificity was 86%, sensitivity was 70% and positive predictive value was 59% (FIG. 5d).

The correlation between circulating miR-21 levels and 11 clinicopathological factors was assessed. Univariate analysis showed that visceral metastasis and lymph node metastasis were significant factors for higher levels of circulating miR-21. However, multivariate analyses showed that visceral metastasis was the only clinicopathological factor significantly correlated to higher levels of circulating miR-21 (FIG. 6).

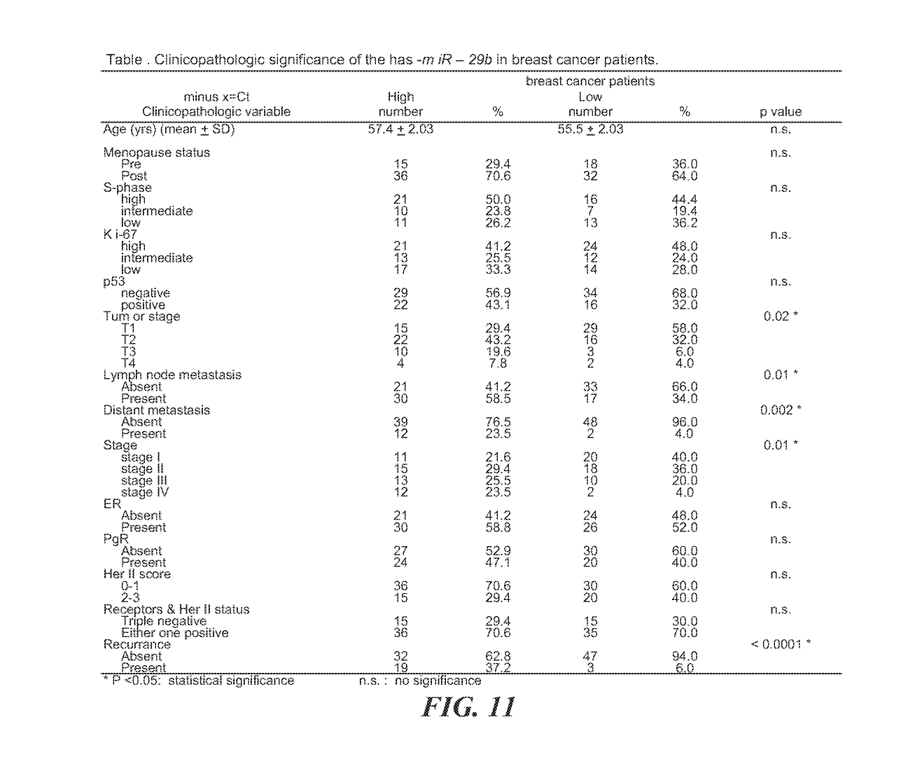

In addition, the correlation between circulating miR-29b levels and 14 clinicopathological factors was assessed. Statistical analysis showed that Tumor stage (i.e., size of tumor), distant or visceral metastasis, lymph node metastasis and AJCC stage were significant factors for higher levels of circulating miR-29b (FIG. 11).

Low Expression of miR-29b Correlates With Higher Survival Rate

To determine miR molecule effect on prognosis, expression of miR-29b expression levels were measured in breast cancer patients that underwent surgical resection of a breast cancer tumor. Patients that were determined to have a high miR-29b expression were more likely to have a poor prognosis (i.e., a low rate of disease free survival) as compared to patients that have a high miR-29b expression level (FIG. 9). Likewise, a patient having high miR-29b expression is more likely to have a good prognosis (i.e., a high rate of disease free survival). These results indicate that miR molecules such as miR-29b are predictive markers of a prognosis or outcome of a cancer.

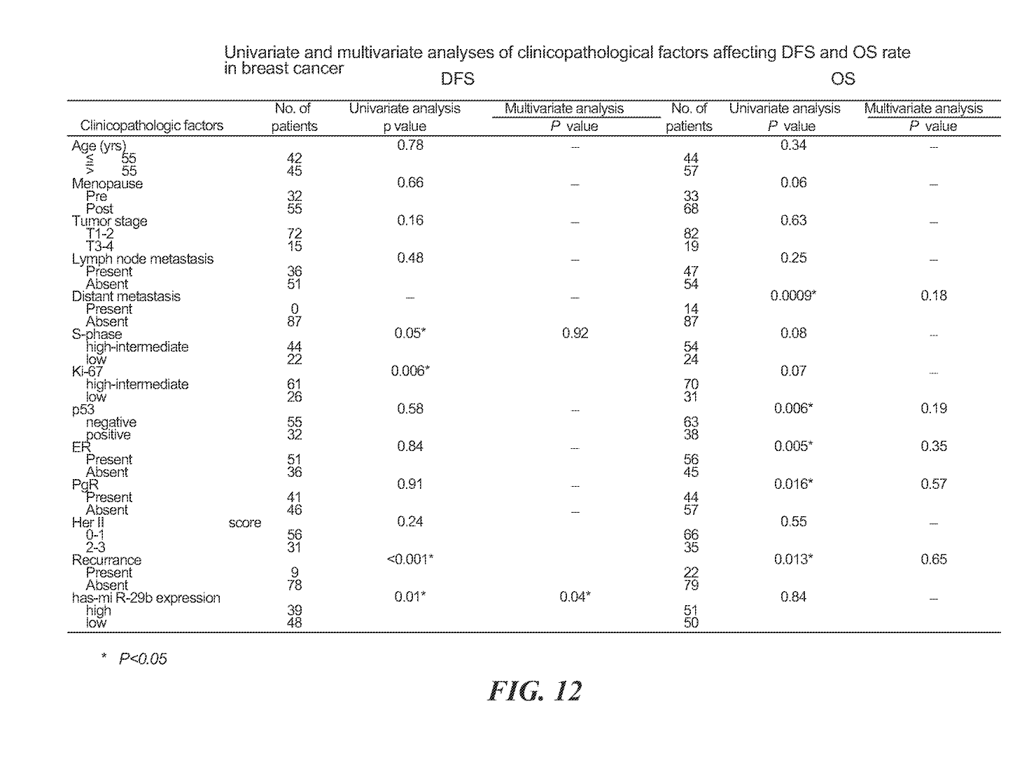

The correlation between circulating miR-29b levels and 11 clinicopathological factors affecting disease free survival (DFS) and overall survival (OS) was assessed. Univariate analysis showed that S-phase, Ki-67, recurrence and miR-29b expression were significant factors for DFS; and distant metastases, p53, ER, PgR and recurrence were significant factors for OS. However, multivariate analyses showed miR-29b expression was the only clinicopathological factor significantly correlated to disease free survival (FIG. 12). Because miR-29b, but not miR-29a or miR-29c showed significant correlation to disease free survival, it is noted that individual members of an miRNA family do not necessarily share the same role as a prognostic or predictive biomarker for a disease or condition.

### EXAMPLE 2

### Clinical Relevance of Serum miR-210 and miR-21 in Melanoma Cancer Patients

Expression levels of miR-21 and miR-210 may be used according to the methods described above to diagnose, prognose and analyze a cancer in a subject. As shown in FIG. 13, plasma expression levels of miR-210 were determined and normalized using an internal standard of miR-16 in metastatic (within 30 days of recurrence, n=43) and normal patients (n=23) A significant difference in the expression ratio of miR-210 to miR-16 (miR-21/miR-16) was found in metastatic melanoma patients as compared to normal patients, indicating that miR-210 can differentially diagnose metastatic cancer and normal or benign conditions (Wilcoxon p=0.0073).

Further, as shown in FIG. 14, plasma expression levels of miR-21 were determined and normalized using an internal standard of miR-16 in Stage III melanoma patients (n=18) and Stage IV melanoma patients (n=20). A significant difference in the expression ratio of miR-21 to miR-16 (miR-21/miR-16) was found in plasma from stage IV melanoma patients (n=20) as compared to stage III melanoma patients (n=18), indicating that miR-21 can differentially diagnose Stage III and stage IV cancer (Wilcoxon p=0.0110).

### REFERENCES

The references listed below and all references cited in the specification above are hereby incorporated in their entirety as if fully set forth herein.

1. Jemal A, Siegel R, Ward E, Hao Y, Xu J, Thun M J. Cancer statistics, 2009. CA Cancer J Clin 2009; 59:225-49.

2. Moss S M, Cuckle H, Evans A, Johns L, Waller M, Bobrow L. Effect of mammographic screening from age 40 years on breast cancer mortality at 10 years' follow-up: a randomised controlled trial. Lancet 2006;368:2053-60.

3. Boyd N F, Guo H, Martin L J, Sun L, Stone J, Fishell E, et al. Mammographic density and the risk and detection of breast cancer. N Engl J Med 2007; 356:227-36.

4. Du T, Zamore P D. microPrimer: the biogenesis and function of microRNA. Development 2005; 132:4645-52.

5. Ambros V. The functions of animal microRNAs. Nature 2004;431:350-5.

6. Bartel D P. MicroRNAs: genomics, biogenesis, mechanism, and function. Cell 2004;116:281-97.

7. Sempere L F, Freemantle S, Pitha-Rowe I, Moss E, Dmitrovsky E, Ambros V. Expression profiling of mammalian microRNAs uncovers a subset of brain-expressed microRNAs with possible roles in murine and human neuronal differentiation. Genome Biol 2004;5:R13.

8. Si ML, Zhu S, Wu H, Lu Z, Wu F, Mo Y Y. miR-21-mediated tumor growth. Oncogene 2007;26:2799-803.

9. Zhu S, Si ML, Wu H, Mo Y Y. MicroRNA-21 targets the tumor suppressor gene tropomyosin 1 (TPM1). J Biol Chem 2007;282:14328-36.

10. Frankel L B, Christoffersen N R, Jacobsen A, Lindow M, Krogh A, Lund A H. Programmed cell death 4 (PDCD4) is an important functional target of the microRNA miR-21 in breast cancer cells. J Biol Chem 2008;283:1026-33.

11. Yan L X, Huang X F, Shao Q, Huang M Y, Deng L, Wu Q L, et al. MicroRNA miR-21 overexpression in human breast cancer is associated with advanced clinical stage, lymph node metastasis and patient poor prognosis. RNA 2008;14:2348-60.

12. Qian B, Katsaros D, Lu L, Preti M, Durando A, Arisio R, et al. High miR-21 expression in breast cancer associated with poor disease-free survival in early stage disease and high TGF-beta1. Breast Cancer Res Treat 2009;117:131-40.

13. Wickramasinghe N S, Manavalan T T, Dougherty S M, Riggs K A, Li Y, Klinge C M. Estradiol downregulates miR-21 expression and increases miR-21 target gene expression in MCF-7 breast cancer cells. Nucleic Acids Res 2009;37:2584-95.

14. Chim S S, Shing T K, Hung E C, Leung T Y, Lau T K, Chiu R W, Lo Y M. Detection and characterization of placental microRNAs in maternal plasma. Clin Chem 2008;54:482-90.

15. Mitchell P S, Parkin R K, Kroh E M, Fritz B R, Wyman S K, Pogosova-Agadjanyan E L, et al. Circulating microRNAs as stable blood-based markers for cancer detection. Proc Natl Acad Sci U S A 2008; 105:10513-8.

16. Chen X, Ba Y, Ma L, Cai X, Yin Y, Wang K, et al. Characterization of microRNAs in serum: a novel class of biomarkers for diagnosis of cancer and other diseases. Cell Res 2008;18:997-1006.

17. Resnick K E, Alder H, Hagan J P, Richardson D L, Croce C M, Cohn D E. The detection of differentially expressed microRNAs from the serum of ovarian cancer patients using a novel real-time PCR platform. Gynecol Oncol 2009;112:55-9.

18. Umetani N, Giuliano A E, Hiramatsu S H, Amersi F, Nakagawa T, Martino S, Hoon D S. Prediction of breast tumor progression by integrity of free circulating DNA in serum. J Clin Oncol 2006a;24:4270-6.

19. Umetani N, Kim J, Hiramatsu S, Reber H A, Hines O J, Bilchik A J, Hoon D S. Increased integrity of free circulating DNA in sera of patients with colorectal or periampullary cancer: direct quantitative PCR for ALU repeats. Clin Chem 2006b;52:1062-9.

20. Takeuchi H, Morton DL, Kuo C, Turner R R, Elashoff D, Elashoff R, et al. Prognostic significance of molecular upstaging of paraffin embedded sentinel lymph nodes in melanoma patients. J Clin Oncol 2004;22:2671-80.

21. Karlen Y, McNair A, Perseguers S, Mazza C, Mermod N. Statistical significance of quantitative PCR. BMC Bioinformatics 2007;8:131.

22. Ng E K, Chong W W, Jin H, Lam E K, Shin V Y, Yu J, et al. Differential expression of microRNAs in plasma of patients with colorectal cancer: a potential marker for colorectal cancer screening. Gut 2009;58:1375-81.

23. Zhu W, Qin W, Atasoy U, Sauter E R. Circulating microRNAs in breast cancer and healthy subjects. BMC Res Notes 2009;2:89.

24. Taylor D D, Gercel-Taylor C. MicroRNA signatures of tumor-derived exosomes as diagnostic biomarkers of ovarian cancer. Gynecol Oncol 2008;110:13-21.

25. Hunter M P, Ismail N, Zhang X, Aguda B D, Lee E J, Yu L, et al. Detection of microRNA expression in human peripheral blood microvesicles. PLoS One 2008;3:e3694.

26. Gibbings D J, Ciaudo C, Erhardt M, Voinnet O. Multivesicular bodies associate with components of miRNA effector complexes and modulate miRNA activity. Nat Cell Biol 2009;11:1143-9.

27. Gastpar R, Gehrmann M, Bausero M A, Asea A, Gross C, Schroeder J A, Multhoff G. Heat shock protein 70 surface-positive tumor exosomes stimulate migratory and cytolytic activity of natural killer cells. Cancer Res 2005;65:5238-47.

28. Harris L, Fritsche H, Mennel R, Norton L, Ravdin P, Taube S, et al. American Society of Clinical Oncology 2007 update of recommendations for the use of tumor markers in breast cancer. J Clin Oncol 2007;25:5287-312.

29. Cristofanilli M, Budd G T, Ellis M J, Stopeck A, Matera J, Miller M C, et al. Circulating tumor cells, disease progression, and survival in metastatic breast cancer. N Engl J Med 2004;351:781-91.

30. Kahn H J, Presta A, Yang L Y, Blondal J, Trudeau M, Lickley L, et al. Enumeration of circulating tumor cells in the blood of breast cancer patients after filtration enrichment: correlation with disease stage. Breast Cancer Res Treat 2004;86:237-47.

31. Taback B, Giuliano A E, Hansen N M, Singer F R, Shu S, Hoon D S. Detection of tumor-specific genetic alterations in bone marrow from early-stage breast cancer patients. Cancer Res 2003;63:1884-7.

32. US Preventive Services Task Force. Screening for breast cancer: U.S. Preventive Services Task Force recommendation statement. Ann Intern Med 2009;151:716-26.

33. Mandelblatt J S, Cronin K A, Bailey S, Berry D A, de Koning H J, Draisma G, et al. Effects of mammography screening under different screening schedules: model estimates of potential benefits and harms. Ann Intern Med 2009;151:738-47.

34. Nicoloso M S, Spizzo R, Shimizu M, Rossi S, Cahn G A. MicroRNAs: the micro steering wheel of tumour metastases. Nat Rev Cancer 2009;9:293-302.

What is claimed is:

1. A method of diagnosing a cancer in a subject, comprising measuring a test level of one or more miR molecules in a biological sample from the subject;
comparing the test level to a control level of the one or more miR molecule; and
diagnosing a subject as having a cancer when the test level is significantly different than the control level.

2. The method of claim 1, wherein the one or more miR molecules are selected from miR-16, miR-21, miR-29b or miR-210.

3. The method of claim 1, wherein the biological sample is a blood sample, a serum sample or a plasma sample.

4. The method of claim 1, wherein the test level and the control level are a mean C_{q} test value and a mean C_{q} control value,

5. The method of claim 4, wherein the mean C_{q} test value and a mean C_{q} control value are normalized by an internal control.

6. The method of claim 1, wherein the cancer is breast cancer or melanoma cancer.

7. The method of claim 1, wherein the test level of the one or more miR molecules is detected by performing a direct reverse-transcription quantitative real-time polymerase chain reaction (RT-qPCR) assay without an RNA extraction step.

8. A method of determining the progression of a cancer in a subject, comprising:
measuring a test level of one or more miR molecules in a biological sample from the subject;
comparing the test level to a control level of the one or more miR molecules; and
differentiating between a locoregional cancer and a cancer that has progressed to a cancer with visceral or distant metastasis when the test level is significantly different than the control level.

9. The method of claim 8, wherein the locoregional cancer is an AJCC stage I-III cancer.

10. The method of claim 8, wherein the visceral or distant metastatic cancer is an AJCC stage IV cancer.

11. The method of claim 8, wherein the one or more miR molecules are selected from miR-16, miR-21, miR-29b or miR-210.

12. The method of claim 8, wherein the biological sample is a blood sample, a serum sample or a plasma sample.

13. The method of claim 8, wherein the test level and the control level are a mean C_{q} test value and a mean C_{q} control value,

14. The method of claim 13, wherein the mean C_{q} test value and a mean C_{q} control value are normalized by an internal control.

15. The method of claim 8, wherein the cancer is breast cancer or melanoma cancer.

16. The method of claim 8, wherein the test level of the one or more miR molecules is detected by performing a direct RT-qPCR assay without an RNA extraction step.

17. A method of determining a prognosis of a subject having a cancer, comprising:
measuring a test level of one or more miR molecules in a biological sample from the subject;
comparing the test level to a control level of the one or more miR molecules; and
determining a prognosis for the subject having a cancer when the test level is significantly different than the control level.

18. The method of claim 17, wherein the prognosis is a poor prognosis or a good prognosis, measured by a shortened survival or a prolonged survival, respectively.

19. The method of claim 18, wherein the survival may be measured as an overall survival (OS) or disease-free survival (DFS).

20. The method of claim 17, wherein the cancer is breast cancer or melanoma cancer.

21. A method of detecting circulating miRNA in a biological sample comprising:
performing a direct RT-qPCR assay without an RNA extraction step on a biological sample from a subject having or suspected of having cancer to detect a level of microRNA.

22. The method of claim 21, wherein the direct RT-qPCR assay comprises mixing the biological sample with a detergent.

23. The method of claim 22, wherein the detergent is Tween 20.

24. The method of claim 22, wherein the detergent is part of a preparation buffer.

25. The method of claim 21, wherein the miRNA is miR-16, miR-21, miR-29b, miR-210 or a combination thereof.

26. The method of claim 21, wherein the level of miRNA is compared to a control level of microRNA to determine the presence or progression of a cancer in the subject.
